(19) United States
(12) Patent Application Publication
(10) Pub. No.: US 2003/0143271 A1
(43) Pub. Date: Jul. 31, 2003

(54) Drug mixture with enhanced dissolution rate

(76) Inventors: Gary D. Ewing, Kalamazoo, MI (US); Michael Hawley, Kalamazoo, MI (US); Martin J. Coffey, Portage, MI (US); Jeffrey E. Price, Middlebury, IN (US); Stephen P. MacMillan, Newton, PA (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/337,583

(22) Filed: Jan. 7, 2003

Related U.S. Application Data
(60) Provisional application No. 60/346,560, filed on Jan. 7, 2002.

(51) Int. Cl. A61K 9/48; A61K 9/22

(52) U.S. Cl. 424/468; 424/452; 514/161

(57) ABSTRACT

A pharmaceutical composition comprises one or more discrete orally deliverable dosage forms, each comprising a poorly soluble coxib component in an amount effective when administered once daily for treatment or prevention of a COX-2 mediated disorder, an aspirin component in a cardioprotective effective amount when administered once daily, and at least one pharmaceutically acceptable excipient; the dosage forms having no substantial barrier to intimate commingling of the coxib and aspirin components. A method of simultaneously treating or preventing a COX-2 mediated disorder and providing cardioprotection comprises orally administering such a pharmaceutical composition to a subject in need thereof.

This application claims priority of U.S. provisional application Serial No. 10/346,560 filed on Jan. 7, 2002.

### FIELD OF THE INVENTION

The present invention relates to a pharmaceutical composition comprising a selective cyclooxygenase-2 inhibitory drug and acetylsalicylic acid, and to therapeutic and/or prophylactic use of such a composition.

### BACKGROUND OF THE INVENTION

Inhibition of cyclooxygenase (COX) enzymes is believed to be at least the primary mechanism by which nonsteroidal anti-inflammatory drugs (NSAIDs) exert their characteristic anti-inflammatory, antipyretic and analgesic effects, through inhibition of prostaglandin synthesis. Conventional NSAIDs such as ketorolac, diclofenac, naproxen and salts thereof inhibit both the constitutively expressed COX-1 and the inflammation-associated or inducible COX-2 isoforms of cyclooxygenase at therapeutic doses. Inhibition of COX-1, which produces prostaglandins that are necessary for normal cell function, appears to account for certain adverse side effects that have been associated with use of conventional NSAIDs. By contrast, selective inhibition of COX-2 without substantial inhibition of COX-1 leads to anti-inflammatory, antipyretic, analgesic and other useful therapeutic effects while minimizing or eliminating such adverse side effects. Selective COX-2 inhibitory drugs such as celecoxib and rofecoxib, first commercially available in 1999, have therefore represented a major advance in the art. These drugs are formulated in a variety of orally deliverable dosage forms.

Acetylsalicylic acid (aspirin) and prodrugs thereof and salts thereof are NSAIDs that have been especially associated with undesirable gastric side effects, including bleeding and/or perforation of the wall of the upper gastrointestinal (GI) tract. Co-administration of aspirin with a selective COX-2 inhibitory drug has generally not been recommended, at least in part because of a desire not to jeopardize the reduced upper GI tract complications offered by the selective COX-2 inhibitory drug by adding a known GI tract irritant, namely aspirin, and because the principal benefits of the aspirin have been perceived to be anti-inflammatory, antipyretic and analgesic activity which can be provided at least as effectively by the selective COX-2 inhibitory drug.

However, in addition to its anti-inflammatory, antipyretic and analgesic effects, aspirin has been reported to provide certain cardioprotective benefits. Attempts to avoid upper GI tract complications while maintaining the beneficial cardioprotective effects of aspirin have involved administration of aspirin in dosage amounts well below the 325 mg typically giving anti-inflammatory, antipyretic or analgesic effect, and/or formulated in a way that modulates contact of aspirin with the wall of the upper GI tract. For example, a dosage amount of about one-fourth of the anti-inflammatory, antipyretic or analgesic dose, i.e., about 81 mg, of aspirin, typically formulated in an enteric coated tablet, is commonly recommended for cardioprotective use with minimal risk of upper GI tract side-effects.

U.S. Pat. No. 6,136,804 to Nichtberger discloses a method for treating, preventing, or reducing the risk of developing acute coronary ischemic syndrome, thrombosis, thromboembolism, thrombotic occlusion and reocclusion, restenosis, transient ischemic attack, and first or subsequent thrombotic stroke by administering an antiplatelet agent in combination with a selective COX-2 inhibitor. Aspirin is identified therein as a suitable antiplatelet agent, and dosage amounts of aspirin of about 75 to about 325 mg/day are proposed.

Greenberg et al. (2000), J. Clin. Pharmacol. 40(12), 1509-1515, reported that the selective COX-2 inhibitor rofecoxib administered in combination with low-dose aspirin did not alter antiplatelet effects of the aspirin in healthy subjects. However, Boers (2001), “NSAIDs and selective COX-2 inhibitors: competition between gastroprotection and cardioprotection,” Lancet 357, 1222-1223, suggested that the gastroprotective benefit of a selective COX-2 inhibitor such as celecoxib over a conventional NSAID was substantially negated in patients taking low-dose aspirin.

Ouellet et al. (2001), “A high level of cyclooxygenase-2 inhibitor selectivity is associated with a reduced interference of platelet cyclooxygenase-1 inactivation by aspirin,” Proc. Nat. Acad. Sci. 98(25), 14583-14588, found that the selective COX-2 inhibitors celecoxib, valdecoxib and rofecoxib had some antagonistic effect on the antiplatelet activity of aspirin, but it was not clear whether the effect was clinically relevant.

Dalen (2002), “Selective COX-2 inhibitors, NSAIDs, aspirin, and myocardial infarction,” Arch. Intern. Med. 162, 1091-1092, concluded that many users of selective COX-2 inhibitors are in an age group at risk of coronary heart disease and recommended concomitant use of low-dose aspirin (80 mg/day).

It would be of benefit to provide, in a single dosage form, both a selective COX-2 inhibitory drug or a prodrug thereof or a salt thereof (a “coxib component”) and acetylsalicylic acid or a prodrug thereof or a salt thereof (an “aspirin component”). The coxib component would provide, with minimal risk of upper GI tract complications, effective treatment and/or prevention of COX-2 mediated disorders such as inflammation; while the aspirin component would provide cardioprotective effect, mediated for example by its antiplatelet activity. Ideally the aspirin component would be present in an amount insufficient to provoke upper GI tract damage.

A formulation providing additive benefits of the coxib component and the aspirin component would have advantage over administration of the two drugs in separate dosage forms, for example in convenience, patient compliance, etc. If a way of formulating the two components could be found that resulted in a synergistic interaction, for example one that enhanced delivery or efficacy of one or both components, a further advantage could be realized. No such formulation approach has hitherto been proposed for a selective COX-2 inhibitor and aspirin.

### SUMMARY OF THE INVENTION

It has now surprisingly been found that when a selective COX-2 inhibitory drug or a prodrug thereof or a salt thereof (a coxib component) having poor solubility in water is coformulated with acetylsalicylic acid or a prodrug thereof or a salt thereof (an aspirin component), in a dosage form that permits the coxib component and the aspirin component to be intimately commingled prior to or upon exposure of the dosage form to an aqueous environment, the coxib component exhibits an enhanced rate of dissolution. Without being bound by theory, it is believed that the enhanced dissolution rate results from formation of a eutectic mixture of the coxib component and the aspirin component. This cannot happen, for example, where the two components are administered separately or where the two components are coformulated in a way that prevents intimate commingling, such as by enteric coating of the aspirin component alone.

Accordingly there is now provided a pharmaceutical composition comprising one or more discrete orally deliverable dosage forms, each comprising a poorly soluble coxib component in an amount effective when administered once daily for treatment or prevention of a COX-2 mediated disorder, an aspirin component in a cardioprotective effective amount when administered once daily, and at least one pharmaceutically acceptable excipient; the dosage forms having no substantial barrier to intimate commingling of the coxib and aspirin components.

In one embodiment the coxib and aspirin components are present in intimate commixture in the dosage form, for example as a thoroughly mixed fine powder blend. Optionally such a blend, referred to herein as a “primary blend”, is itself in commixture with one or more excipients, forming a “secondary blend”. In such a formulation it is preferred that the secondary blend have a microstructure wherein particles of the coxib component remain predominantly in contact with particles of the aspirin component rather than being spatially separated from each other by intervening excipient material. An illustrative process by which such a formulation can be prepared comprises, in the sequence set forth, a first step of triturating the coxib component and the aspirin component in a desired weight ratio to form an API (active pharmaceutical ingredient) blend, a second step of mixing the API blend with one or more pharmaceutically acceptable excipients, and a third step of forming the resulting mixture into a discrete orally deliverable dosage form, for example by molding or compressing to form a tablet or by encapsulating to form a capsule.

In another embodiment the coxib and aspirin components are present in nonintimate mixture in the dosage form, but are disposed therein in such a way that, upon exposure of the mixture to an aqueous medium (e.g., gastrointestinal fluid or a dissolution test medium), the aspirin component begins to dissolve in the aqueous medium and is carried in solution to make contact with the coxib component, resulting in an intimate commingling of the coxib and aspirin components in accordance with the invention. An illustrative process by which such a formulation can be prepared comprises mixing the coxib component, the aspirin component and one or more pharmaceutically acceptable excipients in any order, and forming the resulting mixture into a discrete orally deliverable dosage form, for example as outlined above; except that no combination of excipient and mixing order is used that would result in formation of a barrier between the aspirin component and the coxib component that inhibits commingling of these components upon exposure of the mixture to an aqueous medium.

According to either of the above embodiments, a dissolution-retarding layer (e.g., an enteric coating) can optionally be present enclosing one or more regions of the dosage form (e.g., the entire dosage form or individual pellets or granules within the dosage form), provided that both coxib and aspirin components are present in a desired weight ratio in any such region.

Also provided by the present invention is a method of simultaneously treating or preventing a COX-2 mediated disorder and providing cardioprotection, the method comprising orally administering to a subject in need thereof a pharmaceutical composition as described above, preferably one dosage form being administered once daily.

### BRIEF DESCRIPTION OF THE DRAWINGS

FIG. 1 shows schematic diagrams of dosage forms A-F of the invention and comparative dosage forms G and H not conforming to the invention.

FIG. 2 presents results of a differential scanning calorimetry (DSC) study of various celecoxib/aspirin mixtures by comparison with celecoxib alone and aspirin alone, as described in Example 1.

FIG. 3 presents results of an intrinsic dissolution study comparing a celecoxib/aspirin mixture with celecoxib alone and aspirin alone, as described in Example 2 (“au” means absorbance units).

FIG. 4 presents results of a dissolution assay comparing encapsulated API compositions as described in Example 3.

### DETAILED DESCRIPTION OF THE INVENTION

A pharmaceutical composition of the invention comprises one or more discrete orally deliverable dosage forms. The term “orally deliverable” herein means suitable for administration by mouth, including peroral, sublingual and buccal administration, optionally following dissolution in an imbibable liquid, e.g., water. Preferably a dosage form is suitable for administration per os, whole or broken but without prior dissolution or dispersion in liquid, although liquid can be given to assist swallowing of the dosage form. Any suitable discrete dosage form can be used, including without limitation a tablet (including a variant thereof, such as a caplet), a solid-filled or liquid-filled hard or soft capsule, a lozenge, a separated powder (typically packaged in a single-dose sachet), etc.

Presently preferred dosage forms are tablets and hard capsules. Tablets useful herein typically contain the composition of the invention in compressed form, for example in a form of a compressed granulated mixture. Tablets can be coated or uncoated. Capsules useful herein typically contain the composition of the invention in a form of more or less free-flowing granules and have a wall comprising one or more suitable materials, for example gelatin and/or hydroxypropylmethylcellulose.

Each dosage form comprises a poorly soluble coxib component. The term “poorly soluble” herein means having solubility in water at 20-25° C. not greater than about 10 mg/ml, preferably not greater than about 1 mg/ml, more preferably not greater than about 0.1 mg/ml (100 ppm). The term “coxib” herein embraces all selective COX-2 inhibitory drugs, in particular those having a COX-1/COX-2 selectivity ratio greater than about 10, preferably greater than about 50, more preferably greater than about 100. For the present purpose, selectivity ratio is defined as the ratio of IC₅₀ for COX-1 to IC₅₀ for COX-2, as measured in vitro or in vivo, IC₅₀ being the concentration of a compound that produces 50% inhibition of activity of COX-1 or COX-2. The term “coxib” herein also embraces prodrugs of such selective COX-2 inhibitory drugs, and salts of such drugs and prodrugs.

A preferred coxib useful herein is a compound of formula (I):

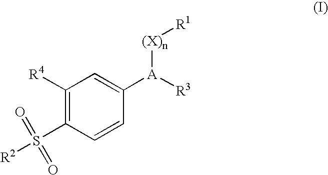

or a prodrug thereof or a pharmaceutically acceptable salt thereof, wherein:

A is a substituent selected from partially unsaturated or unsaturated heterocyclyl and partially unsaturated or unsaturated carbocyclic rings, preferably a heterocyclyl group selected from pyrazolyl, furanonyl, isoxazolyl, pyridinyl, cyclopentenonyl and pyridazinonyl groups;

X is O, S or CH₂;

n is O or 1;

R¹ is at least one substituent selected from heterocyclyl, cycloalkyl, cycloalkenyl and aryl, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is methyl, amino or aminocarbonylalkyl;

R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl, R³ being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and

R⁴ is selected from hydrido and halo.

In one preferred embodiment the coxib is a compound having the formula (II):

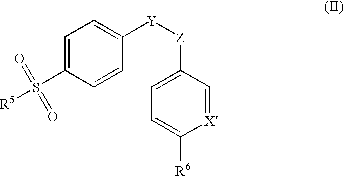

or a prodrug thereof or a pharmaceutically acceptable salt thereof, where R⁵ is a methyl or amino group, R⁶ is hydrogen or a C₁₋₄ alkyl or alkoxy group, X′ is N or CR⁷ where R⁷ is hydrogen or halogen, and Y and Z are independently carbon or nitrogen atoms defining adjacent atoms of a five- to six-membered ring that is optionally substituted at one or more positions with oxo, halo, methyl or halomethyl groups, or an isomer or tautomer thereof. Preferred such five- to six-membered rings are cyclopentenone, furanone, methylpyrazole, isoxazole and pyridine rings substituted at no more than one position.

In another preferred embodiment the coxib is a compound having the formula (III):

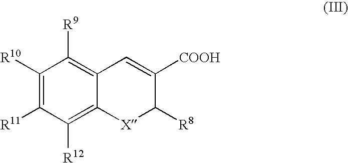

or a prodrug thereof or a pharmaceutically acceptable salt thereof, where X″ is O, S or N-lower alkyl; R⁸ is lower haloalkyl; R⁹ is hydrogen or halogen; R¹⁰ is hydrogen, halogen, lower alkyl, lower alkoxy or haloalkoxy, lower aralkylcarbonyl, lower dialkylaminosulfonyl, lower alkylaminosulfonyl, lower aralkylaminosulfonyl, lower heteroaralkylaminosulfonyl, or 5- or 6-membered nitrogen-containing heterocyclosulfonyl; and R¹¹ and R¹² are independently hydrogen, halogen, lower alkyl, lower alkoxy, or aryl.

A particularly useful compound of formula (III) is (S)-6,8-dichloro-2-(trifluoromethyl)-2H-1-benzopyran-3-carboxylic acid.

In another preferred embodiment the coxib is a 5-alkyl-2-arylaminophenyl-acetic acid or derivative thereof. Particularly useful compounds of this class are 5-methyl-2-(2′-chloro-6′-fluoroanilino)phenylacetic acid and pharmaceutically acceptable salts thereof.

Salts of coxibs or their prodrugs comprise one or more pharmaceutically acceptable counterions. Such salts illustratively include base addition salts having inorganic cations such as alkali metal and alkaline earth metal cations, for example aluminum, calcium, lithium, magnesium, potassium, sodium and zinc, or organic cations prepared from amines such as tromethamine, diethylamine, N,N′-dibenzylethylenediamine, chloroprocaine, choline, diethanolamine, ethylenediamine, meglumine, procaine and the like.

Illustratively, celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 5-methyl-2-(2′-chloro-6′-fluoroanilino)phenylacetic acid and their salts, more particularly celecoxib, valdecoxib, rofecoxib and etoricoxib, are useful in a composition of the invention.

A presently particularly preferred coxib is celecoxib. The invention is illustrated herein with particular reference to celecoxib as the coxib component, but it will be understood that other coxibs can be substituted if desired.

The coxib component is present in each dosage form of a composition of the invention in an amount effective when administered once daily for treatment or prevention of a COX-2 mediated disorder. Suitable dosage amounts can be determined by reference to standard prescribing information for the coxib in question, as set forth for example in Physician's Desk Reference (PDR) and other sources. In the case of celecoxib, a suitable dosage amount will normally be found in a range from about 50 mg to about 400 mg, although greater or lesser amounts can be useful in particular circumstances. Especially preferred celecoxib dosage amounts are about 75 mg to about 300 mg, for example about 100 mg to about 200 mg. Where the coxib is other than celecoxib, suitable dosage amount are those that are therapeutically equivalent to the celecoxib dosage amounts given above.

Each dosage form further comprises an aspirin component, i.e., acetylsalicylic acid or a prodrug thereof or a salt thereof. Salts of acetylsalicylic acid with cations as listed above for coxib salts are illustratively useful, most preferably alkali and alkaline earth metal salts including the calcium salt. Especially preferred is aspirin in its acid form.

The aspirin component is present in each dosage form of a composition of the invention in a cardioprotective effective amount when administered once daily. It is preferred not to exceed the normal full adult dose of aspirin used as an analgesic or antipyretic. Suitable dosage amounts of aspirin in a composition of the invention will normally be found in the range from about 20 mg to about 325 mg, preferably about 40 mg to about 160 mg. Especially suitable is the normal cardioprotective dosage amount of about 80 mg, although in some circumstances lower dosage amounts, for example less than 75 mg, can be useful.

Each dosage form further comprises one or more pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients include, by way of illustration and not limitation, diluents, disintegrants, binding agents, adhesives, wetting agents, lubricants, glidants, crystallization inhibitors, surface modifying agents, substances added to mask or counteract a disagreeable taste or odor, flavors, dyes, fragrances, and substances added to improve appearance of the composition. Such excipients can be solids, semi-solids, liquids or combinations thereof.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; povidone; and the like. Such diluents, if present, constitute in total about 5% to about 99%, preferably about 10% to about 85%, and more preferably about 20% to about 80%, of the total weight of the composition. The diluent or diluents selected preferably exhibit suitable flow properties and, where tablets are desired, compressibility.

Lactose and microcrystalline cellulose, either individually or in combination, are preferred diluents. The use of extragranular microcrystalline cellulose (that is, microcrystalline cellulose added to a wet granulated composition after a drying step) can be used to improve hardness and/or disintegration time of tablets. Lactose, especially lactose monohydrate, is particularly preferred. Lactose typically provides compositions having suitable drug release rate, stability, pre-compression flowability, and/or drying properties at a relatively low diluent cost. It provides a high density substrate that aids densification during granulation (where wet granulation is employed) and therefore improves blend flow properties.

Compositions of the invention optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colorcon™ 1500), clays (e.g., Veegum™ HV), celluloses such as purified cellulose, microcrystalline cellulose, methylcellulose, carmellose and carmellose sodium, croscarmellose sodium (e.g., Ac-Di-Sol™ of FMC), alginates, crospovidone, and gums such as agar, guar, locust bean, karaya, pectin and tragacanth gums.

Disintegrants can be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

Croscarmellose sodium is a preferred disintegrant, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration properties to granulated compositions.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the therapeutic agents to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; polyethylene glycol (PEG); guar gum; polysaccharide acids; bentonites; povidone, for example povidone K-15, K-30 and K-29/32; polymethacrylates; hydroxypropylmethylcellulose (HPMC); hydroxypropylcellulose (e.g., Klucel™); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the composition.

Compositions of the invention optionally comprise one or more pharmaceutically acceptable wetting agents as excipients. Such wetting agents can assist wetting of the poorly soluble coxib, a condition that is believed to improve bioavailability of the coxib component.

Suitable wetting agents include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the composition.

Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the composition.

Magnesium stearate is a preferred lubricant used, for example, to reduce friction between tableting equipment and a granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the composition.

Glidants can be used to promote powder flow of a solid formulation. Suitable glidants include colloidal silicon dioxide, starch, talc, tribasic calcium phosphate, powdered cellulose and magnesium trisilicate. Colloidal silicon dioxide is particularly preferred.

Other excipients such as colorants, flavors and sweeteners are known in the pharmaceutical art and can be used in compositions of the present invention. Compositions of the invention can further comprise pH modifying or stabilizing agents, for example, buffering agents.

Optionally, one or more effervescent agents can be used as disintegrants and/or to enhance organoleptic properties of compositions of the invention. When present to promote dosage form disintegration, one or more effervescent agents are preferably present in a total amount of about 30% to about 75%, and preferably about 45% to about 70%, for example about 60%, by weight of the composition.

An important aspect of the invention is that a dosage form as herein described must have no substantial barrier to intimate commingling of the coxib and aspirin components. Such commingling can occur during formulation, in which case the dosage form as administered has the coxib and aspirin components already in intimate contact with each other. Alternatively, the commingling can occur upon exposure of the composition to an aqueous medium, for example by commencement of dissolution of the aspirin component in gastrointestinal fluid, or in a dissolution test medium.

Exposure to gastrointestinal fluid can occur immediately upon administration, or it can be delayed, for example by provision of an enteric coating around the entire dosage form or around individual pellets or granules within the dosage form, each enteric coated pellet or granule containing both the coxib and aspirin components. It may be found preferable to provide such a coating to minimize release of the aspirin component in the stomach and duodenum, especially in subjects at elevated risk of gastric or duodenal ulceration. It will be recognized, however, that if only the aspirin component is enteric coated, the benefit of the present invention in enhancing dissolution of the coxib component through intimate commingling with the aspirin component will be lost.

It is believed, without being bound by theory, that the enhanced dissolution rate results from formation of a eutectic mixture of the coxib component and the aspirin component. An embodiment of the present invention is a pharmaceutical composition comprising one or more discrete orally deliverable dosage forms, each comprising a poorly soluble coxib component in an amount effective when administered once daily for treatment or prevention of a COX-2 mediated disorder, an aspirin component in a cardioprotective effective amount when administered once daily, and at least one pharmaceutically acceptable excipient; wherein the coxib and aspirin components form a eutectic mixture prior to or upon exposure of the composition to an aqueous medium.

The term “eutectic mixture” is used herein in the broad sense of an intimate mixture of two components at a weight ratio such that the mixture has a lower melting point than would be predicted from the melting point of either component alone. In the case of celecoxib-aspirin eutectic mixtures useful herein, the melting point of the mixture is substantially equal to or lower than that of pure aspirin (around 142° C.) and much lower than that of pure celecoxib (around 162° C.).

Where the coxib component is celecoxib, a suitable weight ratio of coxib to aspirin is about 10:1 to about 1:4, preferably about 8:1 to about 1:2, more preferably about 5:1 to about 1:1. An exemplary weight ratio is about 2.5:1 or about 1.25:1. Where a coxib other than celecoxib is used, a suitable weight ratio is one that is therapeutically equivalent to the above ratios.

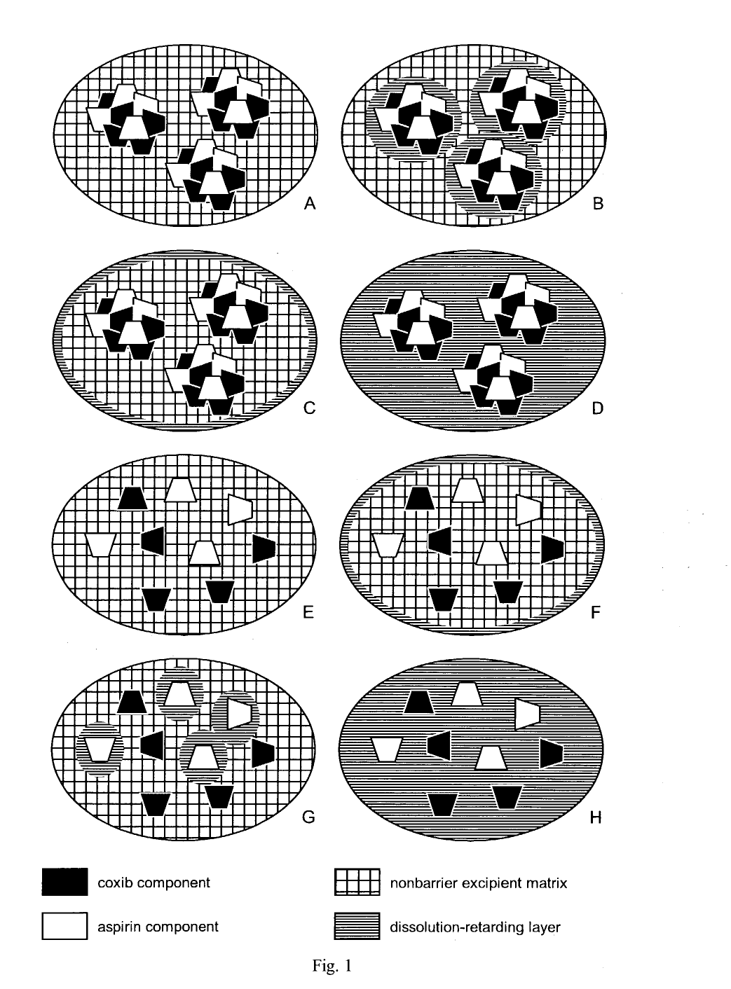

Some examples of formulations having no substantial barrier to intimate commingling of the coxib and aspirin components are illustrated schematically in FIG. 1, A-F.

In A, clusters of drug particles are dispersed in a “nonbarrier excipient matrix”, i.e., an excipient matrix that does not present a barrier to penetration by an aqueous medium. Within each cluster, coxib and aspirin particles are in intimate contact with each other.

In B, similar clusters of coxib and aspirin particles intimately in contact with each other are present, but each cluster is enclosed in a dissolution-retarding layer, for example an enteric coating. Release of both coxib and aspirin components will be delayed, but when an aqueous medium does penetrate into the clusters the benefit of intimate commingling of the two drug components on dissolution rate of the coxib component will be realized.

C and D are variants of B, having respectively a dissolution-retarding outer coat enclosing the entire dosage form, and a dissolution-retarding matrix wherein the clusters are embedded. As in A and B, the coxib and aspirin components are intimately commingled and enhanced dissolution of the coxib component will occur upon penetration of the dissolution-retarding coat or matrix by an aqueous medium.

In E, the coxib and aspirin components are not intimately commingled in the dosage form prior to administration, but instead are independently dispersed throughout a nonbarrier excipient matrix. Upon exposure of the dosage form to an aqueous medium, the aspirin component will immediately begin to dissolve and will be carried in solution to make intimate contact with the coxib component, thereby leading to enhanced dissolution of the coxib component.

F is a variant of E having a dissolution-retarding outer coat that delays penetration of the dosage form by an aqueous medium; however, as soon as the aqueous medium enters the interior of the dosage form the aspirin component will begin to dissolve and make intimate contact with the coxib component as in E.

FIG. 1G and H represent comparative dosage forms not in accordance with the present invention. In G, the aspirin component alone is enclosed in a dissolution-retarding layer, for example an enteric coating. This will not enable the intimate commingling of the coxib and aspirin components necessary for enhanced dissolution of the coxib component. H is a variant of G wherein separate coxib and aspirin particles are dispersed in a dissolution-retarding matrix, again preventing intimate commingling.

Those of skill in the pharmaceutical arts, upon presentation of FIG. 1 and the description of the invention hereinabove, will readily develop methods to make the present compositions. In particular, formulations as depicted in FIG. 1E and F can be made by any standard process of pharmacy that involves mixing of two therapeutically active agents and one or more excipients, followed by a tableting or encapsulating step and, in the case of tablets as depicted in F, a coating step.

Formulations wherein the coxib and aspirin components are intimately commingled in the dosage form prior to administration (for example as depicted in FIG. 1A-D) can be prepared by a process comprising the following steps in the sequence given.

1. The coxib component and the aspirin component are triturated in a desired weight ratio to form an API blend. The term “triturated” herein encompasses any procedure in which the two components are ground or milled together to form a homogeneous powder. On a laboratory scale a mortar and pestle can be used for trituration; on a larger scale any suitable grinding or milling device can be used. The finer the resulting powder, the more intimately will the two components be commingled in the API blend. The API blend is optionally subjected to a compaction step to further enhance contact between coxib and aspirin particles, a procedure described herein as “slugging”.

2. The API blend is then mixed with the desired excipient(s) in any suitable order. Following initial blending of the ingredients, a granulation step is preferably employed to provide a mixture suitable for tableting or encapsulating. Any dry or wet granulation technique known in the art can be used, but a wet granulation step followed by a step of drying the resulting granulate prior to tableting or encapsulating is generally preferred. One or more diluents, one or more disintegrants and one or more binding agents can be added, preferably prior to granulation, a wetting agent can optionally be added, for example in the granulating step, and one or more disintegrants can be added after granulating but before tableting or encapsulating. Disintegrant added prior to granulation becomes intragranular disintegrant, and aids in break-up of granules. Disintegrant added after granulation becomes extragranular disintegrant, and aids in initial separation of granules upon exposure to an aqueous medium. A lubricant is preferably added before tableting. Blending and granulating can be performed independently under low or high shear. A process is preferably selected that forms a granulate that is uniform in drug content, that readily disintegrates, that flows with sufficient ease so that weight variation can be reliably controlled during capsule filling or tableting, and that is dense enough in bulk so that a batch can be processed in the selected equipment and individual doses fit into the specified capsules or tablet dies.

3. The mixture or dried granulate resulting from step 2 is formed into a discrete orally deliverable dosage form, for example by molding or compressing to form a tablet or by encapsulating to form a capsule. Optionally a tablet prepared in this step can be subjected to a further step of coating, for example enteric coating.

The present invention also provides a method of simultaneously treating or preventing a COX-2 mediated disorder and providing cardioprotection to a subject in need thereof. The method comprises orally administering to the subject a pharmaceutical composition as described above. The subject can be nonhuman, for example a domestic animal, but is more typically human.

Compositions of the invention are useful in treatment and prevention of a very wide range of disorders mediated by COX-2, including but not restricted to disorders characterized by inflammation, pain and/or fever. Such compositions are especially useful as anti-inflammatory agents, such as in treatment of arthritis. Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendonitis, bursitis, allergic neuritis, cytomegalovirus infection, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including that following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, scleroderma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic disorders, including without limitation inflammatory disorders such as endophthalmitis, episcleritis, retinitis, iriditis, cyclitis, choroiditis, keratitis, conjunctivitis and blepharitis, inflammatory disorders of more than one part of the eye, e.g., retinochoroiditis, iridocyclitis, iridocyclochoroiditis (also known as uveitis), keratoconjunctivitis, blepharoconjunctivitis, etc.; other COX-2 mediated retinopathies including diabetic retinopathy; ocular photophobia; acute trauma of any tissue of the eye including postsurgical trauma, e.g., following cataract or corneal transplant surgery; postsurgical ocular inflammation; intraoperative miosis; corneal graft rejection; ocular, for example retinal, neovascularization including that following injury or infection; macular degeneration; cystoid macular edema; retrolental fibroplasia; neovascular glaucoma; and ocular pain.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are useful in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemangiomas, angiofibroma of the nasopharynx and avascular necrosis of bone; and disorders of the female reproductive system such as endometriosis.

Such compositions are useful in prevention and treatment of benign and malignant tumors and neoplasia including cancer, such as colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in subjects at risk of FAP.

Such compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for compositions of the present invention are for treatment of rheumatoid arthritis and osteoarthritis, for pain management generally (particularly post-oral surgery pain, post-general surgery pain, post-orthopedic surgery pain, and acute flares of osteoarthritis), for prevention and treatment of headache and migraine, for treatment of Alzheimer's disease, and for colon cancer chemoprevention.

In all of the uses described above, compositions of the invention additionally deliver, simultaneously with the COX-2 inhibitory benefit, a cardioprotective benefit due to the aspirin component. The cardioprotective effect of the aspirin is believed to be related to antiplatelet aggregation activity.

A preferred dosage regimen for a composition of the invention corresponds to once-a-day or twice-a-day administration, but can be modified in accordance with a variety of factors. These include the type, age, weight, sex, diet and medical condition of the subject, the nature and severity of the COX-2 mediated disorder, the presence and severity of risk factors for heart disease, the subject's predisposition to gastric effects of the aspirin component, and other factors. Typically a single dosage form is administered once or twice a day, most preferably once a day.

Suitable daily dosage amounts for the coxib component are, in the case of celecoxib, typically about 50 mg to about 400 mg, or therapeutically equivalent amounts in the case of other coxibs. Greater or lesser amounts can be useful in particular circumstances. Especially preferred daily dosage amounts for celecoxib are about 75 mg to about 300 mg, for example about 100 mg to about 200 mg.

Suitable daily dosage amounts for the aspirin component are typically about 20 mg to about 325 mg, preferably about 40 mg to about 160 mg. Especially suitable is the normal cardioprotective dosage amount of about 80 mg, although in some circumstances lower dosage amounts, for example less than 75 mg, can be useful.

### EXAMPLES

The following examples illustrate aspects of the present invention but are not to be construed as limitations. In the examples “aspirin” means acetylsalicylic acid.

### Example 1

A differential scanning calorimetry (DSC) study was conducted to compare melting point of various celecoxib/aspirin mixtures with celecoxib alone and aspirin alone. DSC scan rate was 10 degrees/minute from 100° C. to 200° C., except for aspirin alone, for which the scan was terminated at about 170° C. Standard crimped pans were used.

Compositions used in the study were as shown in Table 1.

TABLE 1
Compositions used in DSC study

| Composition | Aspirin (mg) | Celecoxib (mg) | Total (mg) |
|---|---|---|---|
| aspirin alone | | none | |
| 21% celecoxib | 3.047 | 0.809 | 3.856 |
| 31% celecoxib | 2.7 | 1.2 | 3.9 |
| 46% celecoxib | 1.466 | 1.237 | 2.703 |
| 65% celecoxib | 1.224 | 2.277 | 3.501 |
| 97.6% celecoxib | 0.102 | 4.190 | 4.292 |
| celecoxib alone | none | | |

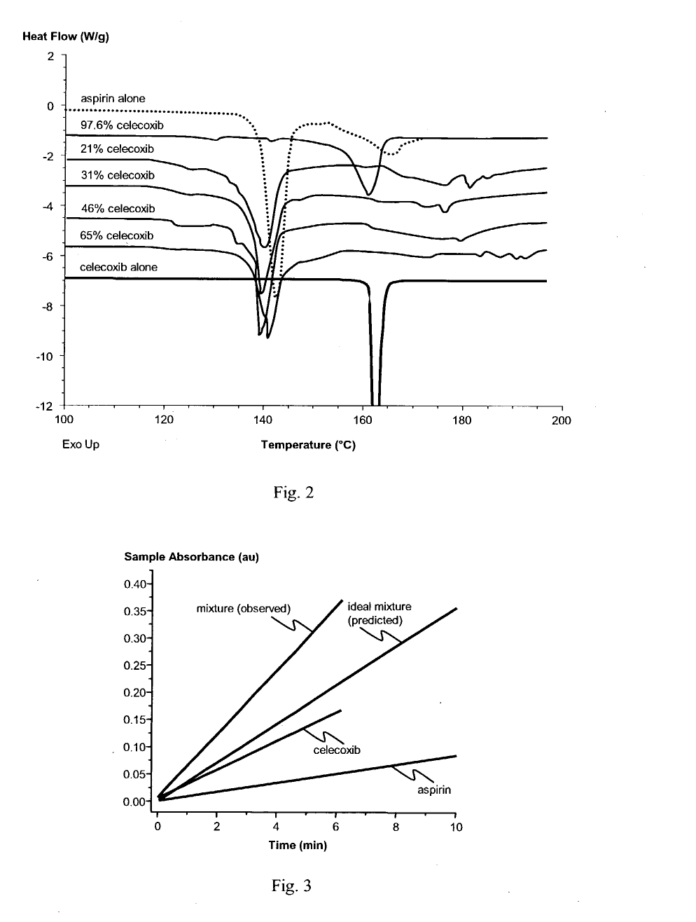

Results of the DSC study are shown in FIG. 2. All tested celecoxib/aspirin mixtures except the 97.6% celecoxib composition exhibited a melting point lower than that of celecoxib alone and equal to or lower than that of aspirin alone, demonstrating unexpectedly that a eutectic mixture was formed between these two compounds.

### Example 2

A rotating disk dissolution study was conducted to compare intrinsic dissolution of a pelleted celecoxib/aspirin dry mixture at a 1:1 weight ratio by comparison with pellets of aspirin alone and celecoxib alone. Pellets were prepared by pressing the material to be pelleted with a 4.5 mm punch and die under 445 N for one minute. The mixture was prepared by triturating celecoxib and aspirin together using a mortar and pestle before pelleting.

Dissolution of celecoxib in water was found to be too slow to be detectable, therefore isopropyl alcohol at 25° C. was used as a dissolution medium. Absorbance of the medium at 254 nm was used as a measure of dissolution. Linear data were obtained for all three compositions, as shown in FIG. 3. Dissolution rate of the mixture was found to be much higher than that predicted for an ideal mixture of the two components.

### Example 3

Three API compositions were prepared for a dissolution study according to USP 24, Apparatus 1 (United States Pharmacopeia 24th ed. (2000), 1941-1943).

Composition A was prepared using celecoxib API that had been slugged to ensure good interparticle contact. The slugged celecoxib API was triturated using a mortar and pestle to provide a homogeneous powder. 100 mg of this powder was filled into each of ten natural transparent Capsugel™ hard capsules, size 1.

Composition B was prepared by first slugging an intimate dry mixture of celecoxib and aspirin in a weight ratio of 100:81 to ensure good interparticle contact, and triturating the resulting slugged mixed API using a mortar and pestle to provide a homogeneous powder. 181 mg of this powder was filled into each of ten natural transparent Capsugel™ hard capsules, size 1.

Composition C was prepared using the same slugged celecoxib API as in composition A. The slugged celecoxib API was triturated using a mortar and pestle to provide a homogeneous powder. Aspirin was separately triturated using a mortar and pestle to a similar particle size as the slugged celecoxib, and the resulting powder was mixed with the celecoxib powder in a celecoxib/aspirin weight ratio of 100:81. 181 mg of the resulting mixture was filled into each often natural transparent Capsugel™ hard capsules, size 1.

A VanKel 7010 dissolution bath fitted with rotating baskets in accordance with USP Apparatus 1, attached to an Alliance 2695D autosampler, was used for the dissolution study. Rotational speed was 100 rpm for 60 minutes, then increased to 250 rpm for a further 60 minutes. The dissolution medium was 0.05M phosphate buffer with 1% polysorbate 80 (Tween™ 20) at pH 6.8. The medium was sampled (10 ml per sample) at 10, 20, 30, 40, 50 and 60 minutes and again at 120 minutes. Samples were analyzed by high pressure liquid chromatography (HPLC).

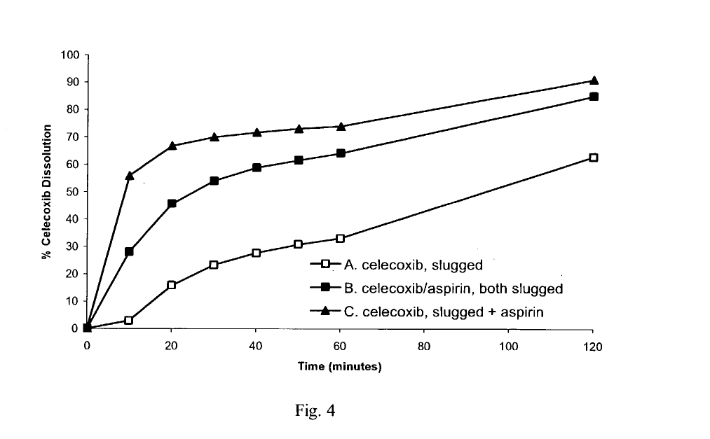

Results are shown in FIG. 4. The celecoxib/aspirin mixtures exhibited greatly enhanced dissolution rate by comparison with the celecoxib alone. It did not appear to be necessary to slug the celecoxib and aspirin together (composition B) in order to obtain this advantage; indeed, simple addition of aspirin to slugged celecoxib (composition C) gave the highest dissolution rate in this study.

What is claimed is:

1. A pharmaceutical composition comprising one or more discrete orally deliverable dosage forms, each dosage form comprising a poorly soluble coxib component in an amount effective when administered once daily for treatment or prevention of a COX-2 mediated disorder, an aspirin component in a cardioprotective effective amount when administered once daily, and at least one pharmaceutically acceptable excipient; wherein the dosage forms have no substantial barrier to intimate commingling of the coxib and aspirin components.

2. The composition of claim 1 wherein said coxib component is a compound having the structural formula 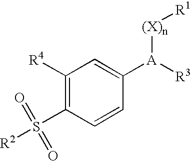
wherein:
A is a substituent selected from partially unsaturated or unsaturated heterocyclic and partially unsaturated or unsaturated carbocyclic rings;
X is O, S or CH₂;
n is O or 1;
R¹ is at least one substituent selected from heterocyclyl, cycloalkyl, cycloalkenyl and aryl groups, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio groups;
R² is methyl, amino or aminocarbonylalkyl;
R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl groups, R² being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio groups; and
R⁴ is selected from hydrido and halo radicals; or a prodrug thereof or a salt thereof.

3. The composition of claim 1 wherein said coxib component is a compound having the structural formula 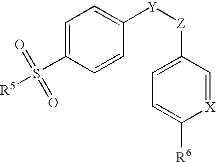
where R⁵ is a methyl, amino or imide group, R⁶ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups; or a prodrug thereof or a salt thereof.

4. The composition of claim 1 wherein said coxib component is a compound having the structural formula 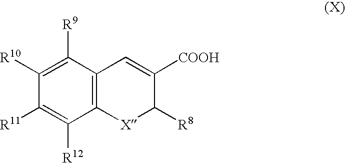
where X″ is O, S or N-lower alkyl; R⁸ is lower haloalkyl; R⁹ is hydrogen or halogen; R¹⁰ is hydrogen, halogen, lower alkyl, lower alkoxy or haloalkoxy, lower aralkylcarbonyl, lower dialkylaminosulfonyl, lower alkylaminosulfonyl, lower aralkylaminosulfonyl, lower heteroaralkylaminosulfonyl, or 5- or 6-membered nitrogen-containing heterocyclosulfonyl; and R¹¹ and R¹² are independently hydrogen, halogen, lower alkyl, lower alkoxy, or aryl; or a prodrug thereof or a salt thereof.

5. The composition of claim 1 wherein said coxib component is selected from the group consisting of celecoxib, deracoxib, valdecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-1-butoxy)-5-[4-(methylsulfonyl)-phenyl]-3-(2H)-pyridazinone, 5-methyl-2-(2′-chloro-6′-fluoroanilino)phenyl-acetic acid and salts thereof.

6. The composition of claim 1 wherein said coxib component is selected from the group consisting of celecoxib, valdecoxib, rofecoxib and etoricoxib.

7. The composition of claim 1 wherein said coxib component is celecoxib.

8. The composition of claim 1 wherein said aspirin component is acetylsalicylic acid.

9. The composition of claim 1 wherein said coxib component and said aspirin component are in intimate contact with each other in the dosage form.

10. The composition of claim 1 wherein said coxib component and said aspirin component become intimately commingled upon exposure of the composition to an aqueous medium.

11. The composition of claim 1 wherein said coxib component is present in an amount therapeutically equivalent to about 50 mg to about 400 mg celecoxib.

12. The composition of claim 1 wherein said coxib component is present in an amount therapeutically equivalent to about 75 mg to about 300 mg celecoxib.

13. The composition of claim 1 wherein said coxib component is present in an amount therapeutically equivalent to about 100 mg to about 200 mg celecoxib.

14. The composition of claim 1 wherein said aspirin component is present in an amount of about 20 mg to about 325 mg.

15. The composition of claim 1 wherein said aspirin component is present in an amount of about 40 mg to about 160 mg.

16. The composition of claim 1 wherein said aspirin component is present in an amount of about 80 mg.

17. The composition of claim 1 wherein said aspirin component is present in an amount of less than 75 mg.

18. The composition of claim 1 wherein said dosage form is selected from the group consisting of a tablet, a capsule, a lozenge and a separated powder.

19. The composition of claim 1 wherein said dosage form is a tablet.

20. The composition of claim 1 wherein said dosage form is a coated tablet.

21. The composition of claim 1 wherein said dosage form is an enteric coated tablet.

22. The composition of claim 1 wherein said dosage form is a capsule.

23. A pharmaceutical composition comprising one or more discrete orally deliverable dosage forms, each dosage form comprising a poorly soluble coxib component in an amount effective when administered once daily for treatment or prevention of a COX-2 mediated disorder, an aspirin component in a cardioprotective effective amount when administered once daily, and at least one pharmaceutically acceptable excipient; wherein the coxib and aspirin components form a eutectic mixture prior to or upon exposure of the composition to an aqueous medium.

24. The composition of claim 23 wherein said coxib component is celecoxib and wherein said coxib component and said aspirin component are present in a weight ratio of about 10:1 to about 1:4.

25. The composition of claim 24 wherein said coxib component and said aspirin component are present in a weight ratio of about 8:1 to about 1:2.

26. The composition of claim 24 wherein said coxib component and said aspirin component are present in a weight ratio of about 5:1 to about 1:1.

27. A process for preparing a pharmaceutical composition, the process comprising:
(a) a step of triturating a coxib component and an aspirin component in a desired weight ratio to form a primary blend;
(b) a step of mixing the primary blend with one or more excipients to form a secondary blend; and
(c) a step of forming the secondary blend into a discrete orally deliverable dosage form.

28. The process of claim 27 wherein said primary blend is subjected to a compaction step to further enhance contact between coxib and aspirin particles prior to mixing with said excipients.

29. The process of claim 27 wherein said secondary blend, prior to being formed into a dosage form, is granulated to provide a mixture suitable for tableting or encapsulating.

30. The process of claim 29 wherein said secondary blend is wet granulated and the resulting granulate is dried prior to tableting or encapsulation.

31. The process of claim 27 wherein said step of forming the secondary blend into a dosage form comprises a tableting step.

32. The process of claim 27 wherein said step of forming the secondary blend into a dosage form comprises an encapsulation step.

33. A product prepared by the process of claim 27.

34. A method of simultaneously treating or preventing a COX-2 mediated disorder and providing cardioprotection, the method comprising orally administering to a subject in need thereof the composition of claim 1.

35. The method of claim 34 wherein one dosage form of said composition is administered once daily.

36. A method of use of a composition of claim 1 in manufacture of a medicament for simultaneously treating or preventing a COX-2 mediated disorder and providing cardioprotection.
